(19) United States
(12) Patent Application Publication
(10) Pub. No.: US 2006/0184029 A1
(43) Pub. Date: Aug. 17, 2006

(54) Ultrasound guiding system and method for vascular access and operation mode

(76) Inventors: Ronen Haim, Ramat Gan (IL); Esther Wischniewski, Berlin (DE); Uri Perets, Beer Sheva (IL); Valery Safran, Cfar Saba (IL)

Correspondence Address: Angenehm Law Firm, Ltd., P.O. Box 48755, Coon Rapids, MN, 55448-0755, US

(21) Appl. No.: 11/331,116

(22) Filed: Jan. 13, 2006

Related U.S. Application Data
(60) Provisional application No. 60/643,168, filed on Jan. 13, 2005.
(60) Provisional application No. 60/650,969, filed on Feb. 9, 2005.

(51) Int. Cl. A61B 8/14

(52) U.S. Cl. 600/443

(57) ABSTRACT

The present invention provides an ultrasound_medical imaging system with integrated devices and functionalities for use in wide range of medical procedures that require vascular and arterial access. The present invention discloses a method for detection, recognition and classification of blood vessels. Said method further calculates the optimal insertion parameters of catheter to the blood vessels. The present invention also exempts the user from handling several procedures simultaneously such as positioning the probe, observing the image or data on the systems screen and locating an accurate location on the surface and thus allowing the user to practice a more accurate procedure.

### CROSS-REFERENCE TO RELATED APPLICATIONS

This application is related to U.S. Provisional Patent Application 60/643,168 filed Jan. 12, 2005 and U.S. Provisional Patent Application 60/650,969 filed Feb. 9, 2005 whose disclosure is incorporated herein in its entirety by reference.

### BACKGROUND OF THE INVENTION

The present invention relates to the field of ultrasonic probes and more particularly to an ultrasonic probe for vascular and arterial access procedures.

Diagnosing human organs using ultrasound scanning is a well known procedure. Ultrasonic transducers based probes direct ultrasonic waves, which travel through a selected biological medium.

Reflections are obtained each time the ultrasonic waves encounter impedance variation interfaces in the biological medium, such as fat and muscle. The returned echoes are received and processed by the imaging system that adds up all scanning lines received from the transducer and provides an image. The number of scanning lines and the depth of examination control the scanning rate. Generally speaking, standard ultrasonic probes use a one dimensional (1D) transducer, wherein the transducer elements are linearly arranged. However, in some probe configurations, multi-dimensional probes (1.5D or 2D) are provided, and the transducer elements are arranged in a matrix, so as to provide 3D steering capabilities.

Conventionally, ultrasonic probes are connected to a system, which is responsible for the processing of electrical signals produced by the probe transducer. The system performs an image capture or rendering operation, using data from the region being scanned, and the obtained images are produced by the synthesizing of information based on a number of different parameters, e.g., the transducer geometry, the number of scanning lines, the depth of examination and the transducer frequency.

The scanning process is usually performed manually. While operating a probe on a surface, the user has to use a screen, which is located far from the probe or the desired scanned area. The user must exercise few simultaneous operations such as surface scanning, monitor screening and ultrasound system control. All these operations require both hands to be used as well as multiple and simultaneous equipment operation.

The operations become rather complex for an insertion of catheters into veins or arteries. Multiple attempts at penetration may result in extreme discomfort to the patient and loss of valuable time during emergency situations. Furthermore, central veins and arteries are often in close proximity to each other. For example, while attempting to access the internal jugular vein, the carotid artery may be punctured accidentally, resulting in severe complications or even death. To prevent complications during catheterization, ultrasonic instruments can be used to determine the location and direction of the blood vessel.

There are several patents that disclose related methods and apparatuses for ultrasonic probes. U.S. Pat. No. 6,733,458 relates to a diagnostic medical ultrasound system having an integrated invasive medical device guidance system. The guidance system obtains image slice geometry and other imaging parameters from the ultrasound system to optimize the guidance computations and visual representations of the invasive medical device and the imaged portion of the subject. Further, the ultrasound system obtains guidance data indicating the relative location, i.e. position and/or orientation of the invasive medical device relative to the transducer and imaging plane to optimize the imaging plane and ultrasound beam characteristics to automatically optimally image both the imaged portion of the subject and the invasive medical device.

Further more, U.S. Pat. No. 6,524,247 provides a method, system, computer program product, and user interface for real-time ultrasonic visualization enhancement of a biopsy needle, in which a wide range of needle positions with respect to the ultrasound probe axis and with respect to the imaged plane are accommodated. Ordinary frames are compounded with special purpose frames, the special purpose frames having transmit and receive parameters adapted to highlight reception of echoes from the biopsy needle. Preferably, an elevation beam width associated with the special purpose ultrasound frames is wider than an elevation beam width associated with the ordinary ultrasound frames. Preferably, the beams of the special purpose ultrasound frames are steered such that they are incident upon the biopsy needle at an increased angle as compared to the angle of incidence for ordinary ultrasound frames. A method for automatically and adaptively determining the depth and orientation of a biopsy needle is also described, whereby beam steering parameters, focus parameters, etc. may be automatically and adaptively computed. The user may optionally provide selected beam steering parameters to the ultrasound imaging system using a simple, intuitive user interface.

Further more, U.S. Pat. No. 6,755,789 provides an apparatus for cannulation of blood vessels, and comprises a sensor assembly including two linear transducer arrays oriented perpendicularly to each other to form a “T” shape to provide substantially simultaneous ultrasound images of at least one blood vessel in a portion of a patient's body in two perpendicular planes. The apparatus may also include one or more Doppler transducer elements to transmit and receive one or more Doppler beams at an incident angle beneath one of the transducer arrays and in alignment therewith to determine blood flow direction and velocity within the at least one blood vessel. The sensor assembly may be disposed within an elongated, flexible, protective sheath and secured to a graphically marked cover to facilitate orientation of the sensor assembly on the patient and guidance of a needle towards a desired target vessel during the cannulation procedure. The cover may also include associated structure to cooperate with a reference location element to place, align and secure the sensor assembly to the patient's skin at a desired location.

None of the patents mentioned above address the needs of detecting, recognizing and classifying blood vessels and integrating a display, a pointing device and a tracking device in the probe housing for simpler and easier usage in medical procedures.

The present invention discloses a method and an apparatus for detecting, recognizing and classifying blood vessels using a display, a pointing device and a tracking device mounted on an ultrasonic probe, for simple and easy usage, exempting the user from handling several procedures simultaneously.

### THE SUMMERY OF THE INVENTION

The object of the invention is to provide an ultrasound_medical imaging system with integrated devices and functionalities for use in wide range of medical procedures that require vascular and arterial access.

The present invention comprises of a method for detection, recognizing and classifying of blood vessels and further more calculates the optimal insertion parameters of catheter to the blood vessels. The present invention also exempts the user from handling several procedures simultaneously such as positioning the probe, observing the image or data on the systems screen and locating an accurate location on the surface and thus allowing the user to practice a more accurate procedure.

Additional guiding is provided for the placement and operation of an invasive device (e.g. a needle, or catheter of any length or type).

The method suggested by the present invention detects, recognizes and classifies blood vessels and calculate the optimal insertion parameters of catheter to the blood vessels. The present invention exempts the user from handling several procedures simultaneously such as positioning the probe, observing the image on the systems screen and locating an accurate location on the surface. Additional guiding is provided for the placement and operation of an invasive device (such as a needle, or catheter of any length or type or any other intrusive tool). Further, a support for invasive device operating nearby vessel, vet required to keep away from vessel (such as in regional anesthesia procedures) is provided as well.

### BRIEF DESCRIPTION OF THE DRAWINGS

These and further features and advantages of the invention will become more clearly understood in the light of the ensuing description of a preferred embodiment thereof, given by way of example only, with reference to the accompanying drawings, wherein—

FIG. 1 is the block diagram of the ultrasound system.

FIG. 2 is the flow chart of the system.

FIG. 3a is the general schematic view from the front of the ultrasonic system with integrated devices.

FIG. 3b is the general schematic view from the rear of the ultrasonic system with integrated devices.

FIG. 3c is the general schematic view of the ultrasonic system with integrated devices when closed.

FIG. 4a is the general schematic view of the ultrasonic system with wireless removable screen.

FIG. 4b is the general schematic view of the ultrasonic system with wired removable screen

FIG. 5 is the general schematic view of the ultrasonic probe with integrated devices.

FIG. 6a illustrates the operation of the probe during scanning and insertion procedure.

FIG. 6b illustrates a structure of acquired ultrasound data in slice representation.

FIG. 7a illustrates a standard ultrasound 2D grayscale image with synthetic enhancements.

FIG. 7b illustrates an optional representation of 3D image with synthetic enhancements.

FIG. 8a illustrates the monitoring of vascular access using an ultrasound probe.

FIG. 8b illustrates optional measurements representation on a synthetic 2D representation.

FIG. 9 illustrates the multi array ultrasonic wave propagation in accordance with present invention;

### DETAILED DESCRIPTION OF THE INVENTION

### Introduction

Ultrasound equipment has developed rapidly over the past 30 years and is now used routinely for numerous medical applications, for example: assessment of arterial stenosis, venous insufficiency and venous thrombosis. Ultrasound images are obtained by holding a probe on the skin surface. An ultrasonic scanner usually has a range of probes with different characteristics, e.g., a linear array probe. This produces a rectangular image which is displayed with the skin surface at the top, the vertical axis showing depth into the body and the horizontal axis showing position along the probe. When imaging blood vessels, the probe can either be placed along the vessel to produce a longitudinal scan or across the vessel to produce a transverse scan. To produce the images, the probe emits short pulses of ultra-sound, and these travel into the body from the probe. Within the soft tissues or at boundaries between them, a small proportion of the ultrasound is scattered or reflected and arrives back at the probe as an echo. The speed of ultrasound in the body is constant (1540 m/s), so the depth of any scatterer or reflector can be found from the time delay from emitting the pulse to receiving the echo. The main pulse continues deeper into the body to be scattered or reflected from deeper structures. When the echoes from one pulse have died down, the next pulse is emitted from a slightly different position along the probe. In this way, it is possible to build up an image of a plane in the body, with depth into the body as the vertical axis and position along the probe as the horizontal axis.

The probe determines the frequency of the ultrasound within the pulses. Higher frequencies give better resolution and more detailed images, but the higher frequency sound loses energy more quickly as it travels through the body so the depth of penetration is less. The operator usually uses as high a frequency as possible. Ultrasound of these frequencies does not travel through air, so a layer of water-based coupling medium is used between probe and skin. The Doppler Effect is a change in the frequency of a wave, resulting from motion of the wave source or receiveror in the case of a reflected wave, motion of the reflector. In medicine, Doppler is used to detect and measure blood flow, and the major reflector is the red blood cell. The Doppler shift is dependent on the frequency, the velocity of moving blood, and the angle between the sound beam and direction of moving blood.

There are several forms of depiction of blood flow in medical Doppler imaging: color Doppler, pulsed Doppler, and power Doppler. Color Doppler provides an estimate of the mean velocity of flow within a vessel by color coding the information and displaying it superimposed on the gray-scale image. The flow direction is arbitrarily assigned the color red or blue, indicating flow toward or away from the transducer, respectively. Pulsed Doppler allows a sampling volume (or gate) to be positioned in a vessel visualized on the gray-scale image, and displays a spectrum, or graph, of the full range (as opposed to the mean velocity, as in color Doppler) of blood velocities within the gate plotted as a function of time. The amplitude of the signal is approximately proportional to the number of red blood cells and is indicated as a shade of gray. Color Doppler provides a global depiction of blood flow in a region and may be used as a guide for the subsequent placement of the pulsed Doppler gate for detailed analysis at a site of potential flow abnormality. Power Doppler, which is not routinely used in arterial Doppler evaluation of the lower extremity, depicts the amplitude, or power, of Doppler signals rather than the frequency shift. This allows detection of a larger range of Doppler shifts and thus better visualization of small vessels, but at the expense of directional and velocity information.

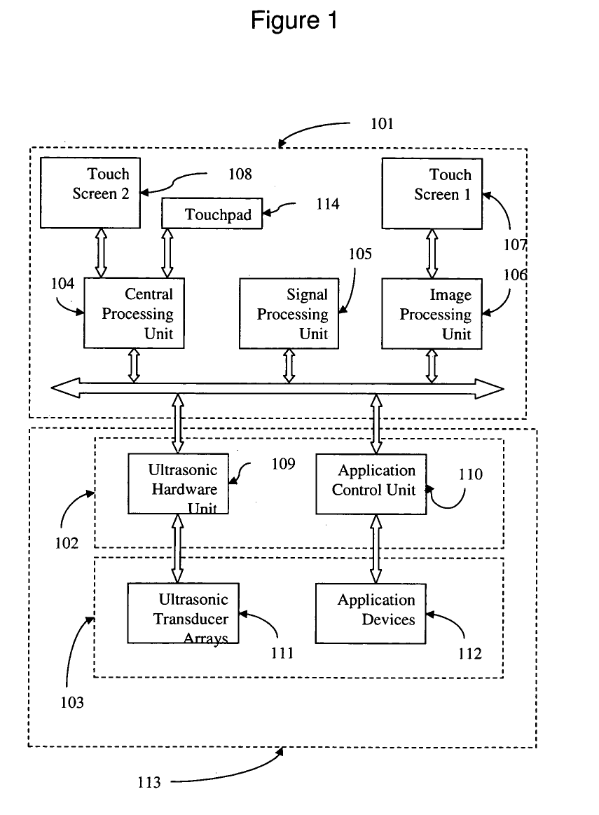

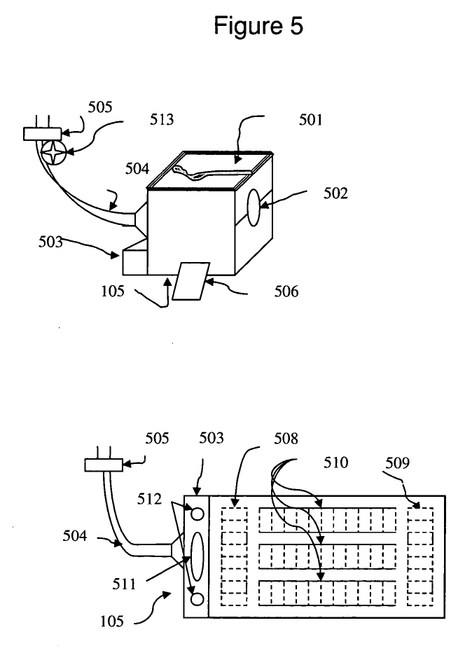

FIG. 1 illustrates the Block diagram of the ultrasonic system. The present invention is comprised of an ultrasonic probe with an integrated display, tracking and pointing devices (103), hardware unit (102) and a processing unit (101). The ultrasonic probe structure is illustrated in FIG. 5.

FIG. 5a illustrates the prolonged cubical shape of the probe which, helps to mount the probe on a human body. The elastic supported arms (506) which are located on both sides of the probe, secures it to the desired surface. According to FIG. 1, the present invention is comprised of multiple transducer arrays (111) with changing configuration, which enables an advanced scanning process, resulting in a 2D or 3D imaging. FIG. 5b illustrates the multiple transducers arrays, which are composed from standard transducers with different configuration. Multiple arrays configuration enables the receiving of 3D sliced image with minimal probe movements, reduces manual operation overhead and eases the standard probe adjustments process, together with a secured probe position to surface, allowing simultaneous operation.

Integrated display shows the data and images provided by the system, so the user can operate the apparatus in accordance to information or image viewed on display. This operational mode can be very helpful in a vascular access operation. The user can operate the probe immediately and in accordance to information obtained directly from the display. An additional accuracy and a comfortable operational environment for medical personal are achieved. Integrated tracking device, such as the optical mouse, allows a better navigation and positioning of the probe on the surface. The pointing device enables the marking of the best possible option of entrance to the blood vessel, making an accurate and precise procedure like vascular puncture possible. FIG. 5b illustrates the multiple transducers arrays, which are divided into three regions: first perpendicular array (508), parallel array (510) and second perpendicular array (509). The probe multiple transducer arrays configuration can consist of either both perpendicular arrays (508+509) or only one of them. Each array is built from two or more transducers.

The Ultrasound system beamformer (102)—combiner (In linear diversity combining, the outputs of two coherent receiving systems are linearly combined to generate the overall system output) deals with each array independently, by switching between the arrays. When an array is switched to the beamformer, all the transducers that are on that array are operational and the system can generate a signal to every transducer within that array. When switched to another array, the Transmit/Receive (T/R) switch must be disconnected first from the previous array and all the transducers on that array are not operational. The system will generate signals to the currently connected array.

The probe (103) emits short pulses of ultra-sound, and these travel into the body from the probe. Within the soft tissues a small, proportion of the ultrasound is scattered or reflected and arrives back at the probe as an echo. The received signals are sent to the central processing unit (104) following pre processing at the beam former. The processed image is then sent back, again via the same connector (505) to the display (501), which is integrated on the probe. The system provides processed images or other information on the display Such as an image of a scanned vascular location.

Integrated tracking system (503) provides the probe positioning and enables sliced indexing, as the location is depended on scanned surfaces.

The Ultrasound hardware (102) illustrated on FIG. 1 includes ultrasonic hardware unit (109) which has beam forming and receiving sequences and probe application control unit (110) which is responsible on operation of all ultrasonic probe (103) application devices (112). The processing unit (101) has a unique structure which contains three processing units, each one responsible for a different task. The central processing unit (104) is used as a task manager that operates all information traffic and operations that must be performed by additional devices. Further more it manages the operation of the touch screen display (108) and receives data from the touchpad device (114). The signal processing unit (105) performs several pre-processing task on the data received from the ultrasound hardware unit (109). The image processing unit (106) uses image processing algorithms and displays the data on the touch screen display (109).

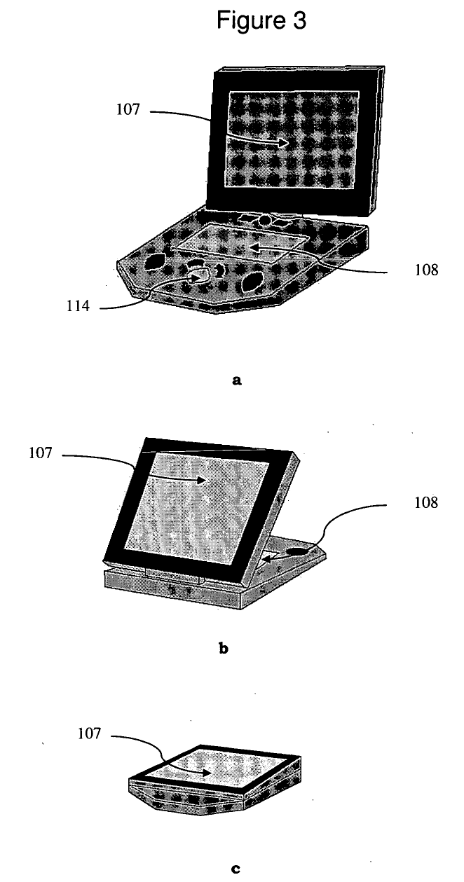

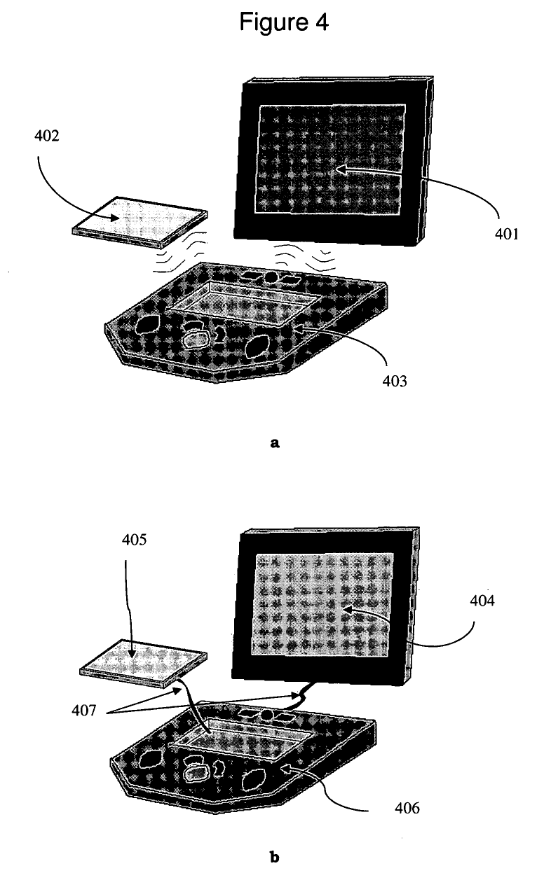

The ultrasound hardware (102) and processing unit (103) are cased in a special designed box as described on FIGS. 3a, 3b and 3c. The touch screen (107) is mounted on a rotating arm enabling the 360° rotation and the screen closing. FIGS. 4a and 4b illustrates the general schematic view of the ultrasonic system with a removable screen.

FIG. 4a illustrates a wireless removable screen. Each touch screen (401 and 402) can be removed and operated separately from the main system case (403) in a wireless manner. Another option is described on FIG. 4b and illustrates the possibility of connecting the screens (404 and 405) by wires (407) to the main system case (406).

The present invention is comprised application of an ultrasonic probe with an integrated display, tracking and pointing devices. Unlike the conventional probe, wherein the scanning probe and display interface are separate units and the user must manipulate the display interface, the probe, the location and the image control, the present apparatus provides the user with many integrated options such as free hand scanning, imaging, data positioning and pointing.

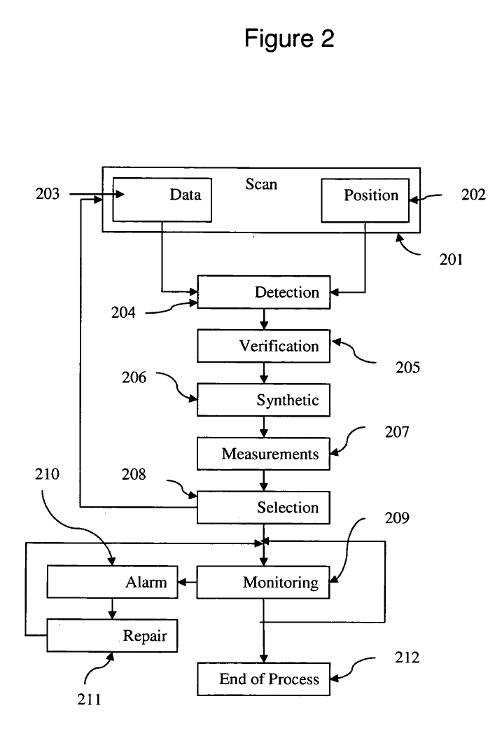

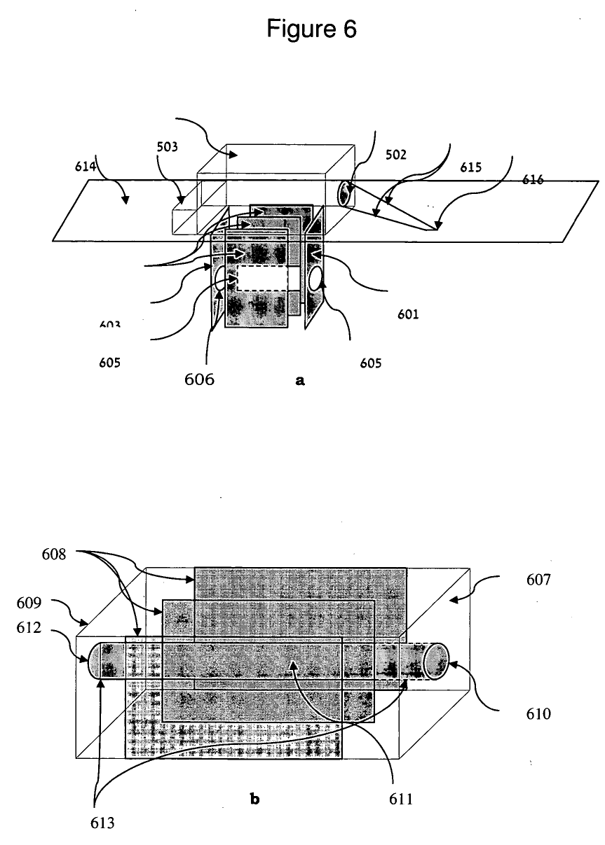

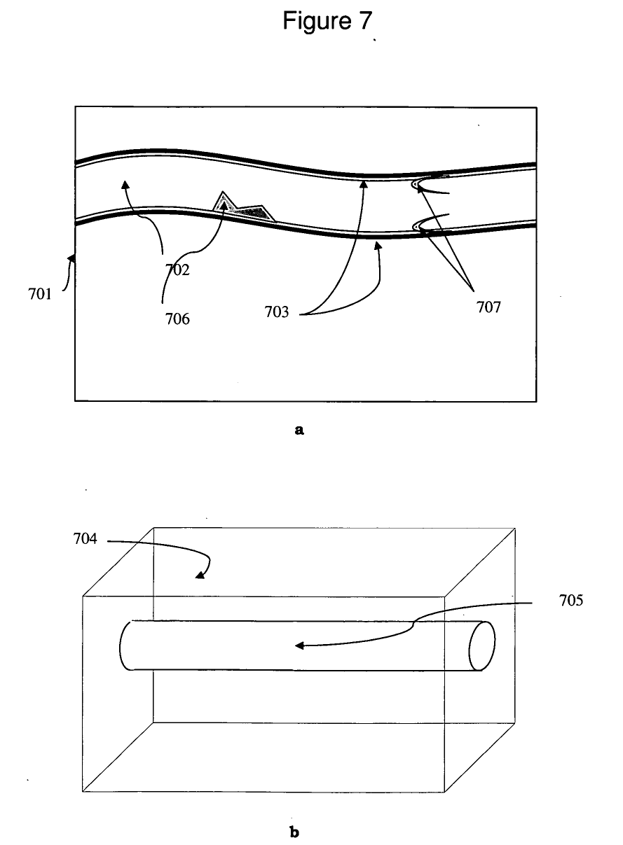

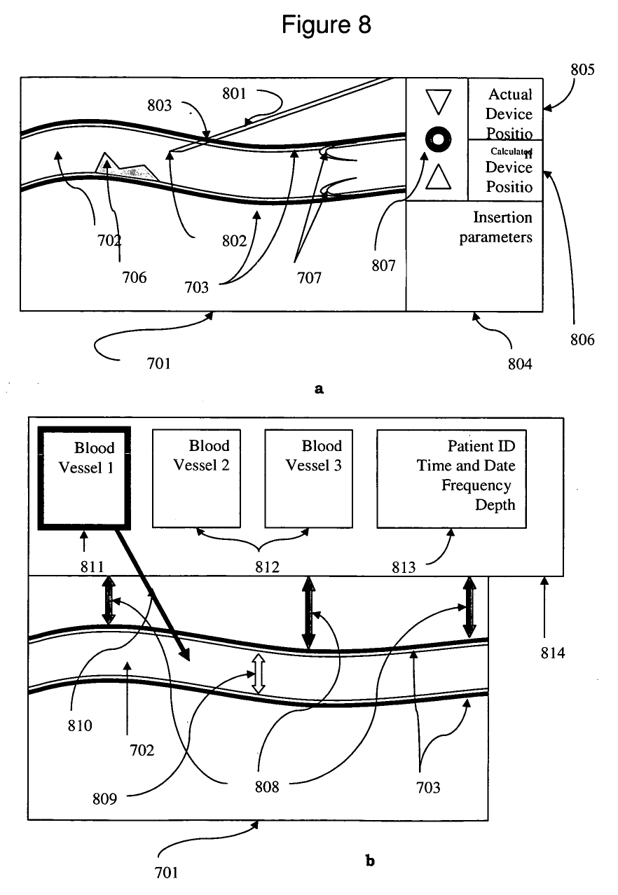

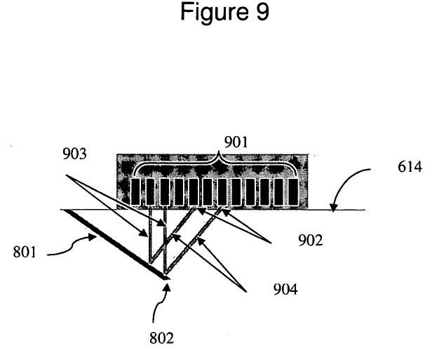

FIG. 2 describes the process of obtaining and displaying the data in a vascular access medical procedure. The ultrasound data (203) and positioning information (202) are obtained during the scan process (201) by the ultrasound device (113). In the Detection phase (204), object segmentation algorithms are applied to the slices with grayscale data. Objects with ultrasonic impedance that resemble blood are categorized as segmented objects. The results of this segmentation are regions with potential blood vessels. For the Verification process (205) power Doppler or pulse Doppler are used to provide information regarding internal areas with blood flow, which are recognized as blood vessels The data obtained on the location of the blood vessels function as a base to the pulse Doppler, which classifies blood vessels as veins or arteries. The information obtained on the blood vessel type serves as a reference to the blood flow velocity and direction. Artery or vein can be classified by the direction of blood flow, or by the relative blood flow velocity. Another method which can be used for blood vessels verification is Color Flow Imaging (CFI). This technique combines Power Doppler Imaging and Pulse Doppler in terms of beamformer operating. As a result, the blood flow inside a blood vessel is detected and its speed and direction are calculated and presented. This method is also effective in blood vessel detection although such calculation requirements will increase the device cost. The ultrasound probe (103) has a pre-determine orientation, blood vessels with blood flow direction outwards of the probe are classified as arteries and blood vessels with an opposite flow direction are classified as veins. In specific situations, blood vessels can be classified using relative blood flow velocity, since blood flow in arteries is different (higher) than in veins. After classifying the blood vessel, an additional search for blood clots (thrombus) and valves is performed. Detected blood clots are identified and monitored and the valves are detected and their status is classified as opened, closed or half-closed as they are monitored during the entire process. The next stage is “Synthetic” (206), where segmented blood vessels are marked or highlighted on the ultrasound image. Once the blood vessels are classified, each one is represented by analytic equitation. This equation applies mathematical manipulation on the blood vessels. Parameterized blood vessels are used for the creation of synthetic 2D images representation (701) as described on FIG. 7a. Synthetic image representation is artificially created and is based on parameterized data with or without representation of additional ultrasound data (any data received from the ultrasound device is described as ultrasound data). Each slice is represented by a synthetic image. The blood vessel (605) is represented on the central slice (608) as an object (611). Synthetic representation (702) of a blood vessel (611) is shown on the composed 2D image (701). The boundaries (703) of the blood vessel (702) are color highlighted, representing the blood vessel type: red for artery and blue for vein. Another optional form of synthetic representation is the blood vessel background color highlighting, in accordance to the blood vessel type. Blood clot (706) is also highlighted and when changing position inside the blood vessel an alarm indicates the move and the new position is being highlighted. Further more, the valve (707) inside the blood vessel (702) is highlighted by different colors according to its status (opened, closed and half closed). When creating a composed 3D image, the composed 2D images are placed in a slice formation (FIG. 6b). Algorithms of interpolation are used to create solid semi-transparent composed 3D image (704) and omitted areas (613) together with apparent areas (610, 611 and 612), are reconstructed as a 3D model of a blood vessel (705). When more than one composed 3D image is created, the set of images are arranged in consecutive order according to their position. In this case topography 3D view of scanned area is created.

In the “Measurements” phase (207), mathematical calculations of a blood vessel parameters and position are performed using parameterized representation of blood vessels (diameter, depth, etc.). Parameters window with the blood vessel's parameters (811), such as diameter and depth, is shown on the information panel (814) together with some standard ultrasound data (813) and an arrow (810) indicates (811) the relevant blood vessel's image (702). Additional blood vessels data are shown on different areas (812). Different arrows (808 and 809) indicate visually the parameters of the blood vessel (702) (diameter and depth accordantly). The measurements are indicated on the synthetic 3D representation similarly to the synthetic 2D representation.

The process of the blood vessel “Selection” (208) is based on a pre-choose or custom scenario, which defines the best type of blood vessel suited for vascular access operation. Blood vessels depth and diameter also play an important role in the selection procedure. Once the blood vessel is selected, insertion parameters are calculated based on access/insertion factors, for example, the angel of insertion, which is based on the tissue surrounding the blood vessel, the point of puncturing the vessel and the point of the invasive device access based on all this calculations. If the ultrasonic probe (103) is not located above a selected part of a blood vessel, a special navigation mode is initiated. In this mode the LCD screen (501), which is mounted on the ultrasonic probe (103), shows the graphic information, which helps navigate the probe to the exact place above the selected part of a blood vessel. After reaching the exact position over the selected part of a blood vessel, the ultrasonic probe (103) is attached to the surface (614) using elastic supported arms (506). A pointing device (502) emits a laser beam (615) to indicate the vascular access optimal insertion point (616). In the case of an absent blood vessel, suited for vascular access, the system will return to the “Scanning” phase (201) and the previously described process will be performed again from the beginning.

The next phase is “Monitoring mode” (209), which monitors the invasive access operation at real time. Ultrasound beam is focused on the boundaries of the blood vessel for obtaining best resolution in these areas and providing best monitoring conditions. Once an invasive device (801) is seen on the synthetic 2D representation (701), an additional focusing is performed and the data regarding the device front edge (802) position is calculated (e.g. a needle tip). Ultrasound waves (903) are emitted by the ultrasound transducers (901) mounted probe (103) to the tissue (905). A portion of those waves reaches the invasive device (801) and the transducer (902) receives the returning waves (903). This process makes it possible to obtain the exact position of the invasive device without any additional noise. The data regarding the invasive device position (805) is shown in an opposite manner to the pre-calculated insertion data (806). This data supports manual verification of the invasive device (801) propagation status. The data can be shown on image (701) in term of symbol, text or graphical format. Additional insertion process data is shown on another panel (804). Visual indicator (807) integrates visualization for the insertion process status of invasive device. In contrary to numeric data representation, when data understanding is required, the described technique provides a fast understanding of the access process status. When the invasive device progressed within the acceptable operation parameters, the process mode is classified as safe and the visual indicator (807) lights up or indicates the safe status. If the invasive device exceeds the acceptable operation parameters, an alert mode (210) is triggered and lights the alarm status. The highlighted pointer shows the directional change required in order to amend the path and to return to acceptable operation parameters. In case of a hazardous situation, an “Alarm mode” (210) is triggered and the user is informed about the situation. In the “Repair mode” (211) a possible solution is displayed. When insertion parameters return to normal, the process returns to “Monitoring mode” (209). The “Alarm mode” is divided to two states: “Attention” and “Alarm”, according to the situation and consequences. For example, a hazardous situation which is classified as “Attention” is when an invasive device tip (802) is close to the point of the blood vessel insertion point (803). An example for the “Alert” is when an invasive device escapes the insertion course and is inserted below the blood vessel. In the same way, two side slices (608) are monitored for the prevention of the blood vessel side lumen puncture. Valves (707) in a blood vessel (702) are also monitored and if an invasive device (801) is close to it, the “Alarm mode” (210) is triggered. Further more, if a blood clot appears on the invasive device or its surroundings, the “Alarm mode” (210) is also triggered.

When the entire process is completed, a special post-processing analyses mode (212) is initiated. In this mode, data is shown with a projection of the invasive device insertion on the synthetic 3D representation (704). This mode provides some tools for the analysis of the data collected during the entire process.

According to further improvement of the present invention it is suggest that the system will be operated by voice commands using speech recognition modules.

The described process can be performed by using ultrasound probe of any configuration. Different configuration of said probe may involve some limitation on described process or result partial process functionality. Examples of said different probe configuration can be: curved probe, linear probe with position device or others.

What is claimed is:

1. A method for blood vessel imaging, detection recognition and selection, said method comprising of:
a. receiving an vascular imaging and positioning information captured by ultrasound hardware;
b. processing of received ultrasound data for detection and recognition of blood vessels.
c. measuring and calculating of blood vessels dimensions and properties.
d. Creating synthetic representation of composite image of the blood vessel comprised of the ultrasound image and supplemental graphical elements and effects;

2. The method of claim 1 further comprising the steps of: selecting of blood vessel suitable for vascular access and providing navigation information for vascular access support for invasive device

3. The method of claim 2 further comprising the step of monitoring of insertion process of the invasive device;

4. The method of claim 3 wherein the monitoring include blood vessel movement, position change or size variation due to expansion or contraction.

5. The method of claim 1 wherein measurements of the blood vessel include: the diameter, depth and distance between the vessels.

6. The method of claim 1 further comprising the step of motion detection utilizing Doppler effect for measuring blood flow velocity and direction.

7. The method of claim 6 wherein the Doppler technique include one or any of the following: Power Doppler Imaging with Pulse Doppler or Color Flow imaging.

8. The method of claim 1 wherein the ultrasound data acquisition is received from at least one linear arrays mounted on ultrasound probe.

9. The method of claim 1 further comprising the step of indexing of ultrasound data according to received position information, wherein each slice keeps his index according to its physical position inside of the probe and relative probe position.

10. The method of claim 1 wherein the synthetic representation include topological view of scanned area.

11. The method of claim 1 wherein the synthetic representation is a 2D or 3D image or combination.

12. The method of claim 1 wherein the graphical elements and effects include marking, coloring, pointing, guiding, adding explanatory images or text.

13. The method of claim 1 wherein processing of the ultrasound image include valve detection inside blood vessel.

14. The method of claim 1 wherein processing of the ultrasound image includes locating of blood clots.

15. The method of claim 2 further comprising the step of providing insertion parameters calculation, including insertion point calculation, insertion trajectory calculation, said calculation based on blood vessels properties measurements.

16. The method of claim 2 further comprising the step of illuminating specific points at the target vessel by a laser pointing device in accordance with insertion parameters calculations.

17. The method of claim 1 further enabling to operate the imaging process by voice commands using speech recognition modules;

18. The method of claim 1 wherein the processing of detection or recognition or both is based on pattern recognition or edge detection or both.

19. The method of claim 1 wherein a system for image guided vascular access is introduced.

20. The method of claim 9 wherein the positioning motion registration sensor is mounted on a probe.

21. The method of claim 1 wherein vessel verification is based on Power Doppler Imaging or Pulse Doppler or Color Flow Imaging.

22. The method of claim 1 wherein Imaging technique compromises automatic or manual operation

23. The method of claim 1 wherein synthetic image compromises grey ultrasound and vessel segmentation data.

24. The method of claim 23 wherein 3D synthetic images compromises image reconstruction of 2D slices.

25. The method of claim 23 wherein synthetic images compromises topological view of 3D image reconstruction technique.

26. The method of clam 5 wherein automatic measurements are performed on synthetic and non synthetic images.

27. The method of claim 13 wherein valve detected, includes one or any of the following detection: Open, close or partly close.

28. The method of claim 14 wherein blood clots detected, includes one or all of the following detection, location, measurements or monitoring

29. The method of claim 1 wherein further compromising of blood vessel selection, includes automatic vessel selection best suitable for insertion.

30. The method of claim 1 wherein further compromising of blood vessel selection, includes voice manual command by user for an identified vessel of choice.

31. The method of claim 1 wherein further compromising of blood vessel selection, includes a screen pointing or touching selection command by user for an identified vessel of choice or location.

32. The method of claim 1 wherein measuring and calculating of blood vessels dimensions and properties, includes one or more of the following calculations: insertion point, insertion trajectory, trajectory margins and custom scenarios.

33. The method of claim 3 wherein includes calculated measurements on synthetic or ultrasound image

34. The method of claim 4 wherein monitoring includes alignment and navigation of probe over blood vessel/s

35. The method of claim 32 wherein laser pointer device compromises the location of insertion point

36. The method of claim 1 wherein insertion navigation parameter are calculated and recalculated against data collected during insertion

37. The method of claim 36 wherein accurate insertion navigation parameter are provided to the user.

38. The method of claim 37 wherein parameters includes invasive device propagation path, margins, angel of insertion, distance to blood vessel, inner vessel measurement, as well as ultrasound image/s and supplemental graphical elements, symbols and effects;

39. The method of claim 38 wherein captured invasive device tip image is captured and mixed with blood vessel image.

40. The method of claim 37 wherein attention mode alerts of conflict within the set of parameters or close to blood vessel wall or when invasive device is sided away from trajectory.

41. The method of claim 40 wherein reverse attention mode includes an option alerting when an invasive device is positioned a short distance or pre selected distance from a vessel.

42. The method of claim 1 wherein at least one LCD is mounted on a probe for image and data display

43. The method of claim 1 wherein at least one LCD is mounted on a system for image and data display
